(19) United States
(12) Patent Application Publication
(10) Pub. No.: US 2023/0181315 A1
(43) Pub. Date: Jun. 15, 2023

(54) ASSEMBLY FOR A CLOSURE DEVICE WHICH IS IMPLANTABLE IN THE SUPERIOR OR INFERIOR VENA CAVA OF A HUMAN BODY IN A MINIMALLY INVASIVE MANNER, AND TRICUSPID VALVE PROSTHESIS WHICH IS IMPLANTABLE IN A MINIMALLY INVASIVE MANNER

(71) Applicant: Immanuel Albertinen Diakonie GmbH, Hamburg (DE)

(72) Inventor: Christian BUTTER, Berlin (DE)

(73) Assignee: Immanuel Albertinen Diakonie GmbH, Hamburg (DE)

(21) Appl. No.: 17/926,211

(22) PCT Filed: May 18, 2021
(86) PCT No.: PCT/DE2021/100441

§ 371 Date: Nov. 18, 2022

(30) Foreign Application Priority Data
May 19, 2020 (DE) 10 2020 113 585.8

(51) Int. Cl. A61F 2/24

(52) U.S. Cl. CPC A61F 2/2403; A61F 2/2412

(57) ABSTRACT

A minimally-invasive implantable closing device (1) in the superior or inferior vena cava of a human body, with a valve device (6) and an anchoring device (7). The valve device (6) has closing elements and a support structure. The closing elements extend flat over a respective joint surface and in each case can be moved between a closed position, in which the closing elements together close a valve opening, and an open position, in which a flow is released through the valve opening. Further, a minimally-invasive implantable tricuspid valve prosthesis is provided.

The invention relates to an arrangement of a minimally-invasive implantable closing device as well as a minimally-invasive implantable tricuspid valve prosthesis in the superior or inferior vena cava of a human body.

### BACKGROUND

A minimally-invasive implantable mitral or tricuspid valve prosthesis is known from the document EP 3 231 393 A1. For positioning and attaching, the valve prosthesis is securely connected to a self-expanding, broad-meshed stent, which is adapted in its shape to a three-dimensional measured geometry of a patient's left or right chamber and after implantation rests on the inside wall of the chamber.

### SUMMARY

The object of the invention is to indicate an arrangement of a minimally-invasive implantable closing device as well as a minimally-invasive implantable tricuspid valve prosthesis in the superior or inferior vena cava of a human body, which device and prosthesis can be securely located at the site of the implantation and ensure a secure opening and closing.

For a solution, an arrangement of a minimally-invasive implantable closing device in the superior or inferior vena cava of a human body is provided. The coordinate claim 15 relates to a minimally-invasive implantable tricuspid plate prosthesis. Additional configurations are the subject matter of dependent subclaims.

According to one aspect, an arrangement of a minimally-invasive implantable closing device in the superior or inferior vena cava of a human body is provided and has the following: a valve device, having closing elements, which extend flat over a respective joint surface, and in each case can be moved between a closed position, in which the closing elements together close a valve opening, and an open position, in which a flow is released through the valve opening, and a support structure, which is arranged in the area of the valve opening and is formed with support elements and at least one indentation. In the closed position, the closing elements rest at least partially on the support elements, and in the open position, they are separated at least partially from the support elements and thus release the indentation to allow flow. In addition, the arrangement has an anchoring device that is formed with an anchor and is set up to anchor the valve device in the area of the superior or inferior vena cava adjacent to the vein opening in the right chamber of the heart.

According to another aspect, a minimally-invasive implantable tricuspid valve prosthesis is provided with such an arrangement.

The proposed closing device can be securely attached at the implantation site by means of the anchoring device. The valve device with the closing elements ensures a reliable opening and closing in order to prevent or permit the flow.

In a possible configuration, the valve opening is designed as a round or circular valve opening.

The support structure can be designed to extend over a part of the surface of the valve opening or over its entire surface.

The closing elements can be designed as non-surface-rigid closing elements, which (optionally in addition) bend in order to swing open into the open position, so that the flow is released. In an alternative embodiment, one or more of the closing elements is/are essentially surface-rigid. In this configuration, the surface-rigid closing element pivots between the closed and the open positions, essentially maintaining the surface shape of the closing element that is present in the closed position. To ensure movability, in particular pivotability, for example, a hinge device can be provided.

The indentation can be designed with an indentation in the area of the center of the valve opening. In addition to the indentation, one or more other indentations can be provided in the support structure. In one or more of the indentations, a sieve insert can be provided, which insert is arranged to be detachably or non-detachably connected in the area of the assigned indentation on the support structure.

Adjacent closing elements can overlap in edge-side sections at least in the closed position, and the support elements can extend at least along the overlapping edge-side sections. The support elements can also optionally run outside of the overlapping areas in order to support the closing elements even outside of the overlapping areas.

The valve device can be designed as a one-way valve. This means that the valve device passes in a flow direction and blocks in the opposite direction, which is achieved by means of the closing elements resting on the support structure in the closed position, which elements can be at least partially raised from the support structure in the flow direction in order to release the flow.

The closing elements can in each case be formed to comprise the sector surface, wherein adjacent closing elements overlap along the radii laterally bounding the sector surface. Thus, the closing elements are comparably arranged in top view in the shape of a pie slice. In the area of a radial inside tip section, in which the radii laterally bounding the sector surface intersect, the sector surface can be rounded. Also, it can be provided that in the area of the tip, a radial inside end section of the sector surface is cut off. The radial inside tip sections of the sector surfaces of the closing elements can overlap in the area of the indentation. An overlapping area that is formed here can have a size (expansion) of approximately 1 mm to approximately 4 mm, alternatively approximately 1 mm to approximately 2 mm, in at least one direction.

The closing elements can in each case comprise a sector of at least approximately 90 degrees. In this embodiment, four closing elements can be provided in order to close together the valve opening in the closed position. This means that at least one or all closing elements comprise(s) a sector of somewhat more than 90 degrees in order to form overlapping areas running along the radii. The respective sectors can comprise an angle of at most approximately 120 degrees to approximately 130 degrees.

The closing elements can be fastened at least in sections along an outside circumferential edge in such a way that a section of the closing element that is radially inside relative to the circumferential edge can swing open into the open position and can thus release the flow. The fastening of the closing elements along the outside circumferential edge can be done in a detachably-connected or non-detachably-connected manner. For non-detachable connection, for example, a joint connection can be provided, for example by means of gluing or welding. In one configuration, the closing elements can be molded in one piece onto the anchoring device and/or the support structure, for example by means of 2-component injection molding. In this case, the anchoring device and/or on the support structure can be made from a plastic hard component, and the closing elements can be made from a plastic soft component. The fastening of the closing elements along the outside circumferential edge can comprise a circular arc of approximately 60 degrees to approximately 110 degrees, alternatively a circular arc of approximately 90 degrees to 110 degrees, for example approximately 105 degrees. If the closing elements are formed as sectors, corner areas in which the outer circular arc pushes open the laterally bounding radii can be designed not to engage with the fastening.

The indentation of the support structure can have a flow nozzle. In this or other configurations, the indentation can be arranged essentially centered in the valve opening. A nozzle surface that runs crosswise to the stream or flow direction of the valve device can be arranged essentially parallel to the valve opening surface that runs crosswise to the stream or flow direction. The nozzle surface can lie essentially in the same plane as the valve opening surface. As an alternative, the nozzle surface in the flow direction is arranged offset relative to (separated from) the valve opening surface. The offset can be, for example, approximately 5 mm to approximately 12 mm, alternatively approximately 7 mm to approximately 10 mm, for example approximately 8 mm to approximately 9 mm. In this or other embodiments, the indentation can be formed with a diameter of approximately 2 mm to approximately 6 mm, alternatively with a diameter of approximately 4 mm to approximately 5 mm. The flow nozzle can be designed in a comparable way.

The support structure can have support elements that extend in a star shape, for example along circle radii of the valve opening. The support elements that extend in a star shape and that are designed, for example, as support carriers can run to an edge of the support structure starting from the indentation or the flow nozzle. The support elements that are arranged in a star shape can be arranged in a radially circumferential manner at essentially equal intervals, for example at a distance of approximately 90 degrees to approximately 120 degrees.

The support elements that are designed as, for example, carriers can have in cross-section a shape that tapers to form a support surface, wherein in the area of the support surface, the closing elements come to rest in the closed position.

In a top view looking down at the valve opening, the support elements can have a width of approximately 1 mm to approximately 5 mm, alternatively of approximately 1 mm to approximately 3 mm, for example approximately 1.5 mm to approximately 2.5 mm. If the support elements are designed as carriers, in this respect a carrier width is provided in a top view looking down at the valve opening.

The support structure can support the closing elements in the closed position in an essentially two-dimensional flat plane. In this embodiment, the opening surface of the indentation can lie crosswise to the flow or stream direction essentially in the plane of the valve opening surface. For the closing elements, a flat support surface is provided by means of the support structure.

The support structure can support the closing elements in the closed position in a curved three-dimensional plane. For the closing elements, a three-dimensional support surface is provided by means of the support structure. In this configuration, the support structure can be formed with a bell shape, so that a bell-shaped support surface is provided. It can be provided that the opening surface of the indentation or the flow nozzle in the flow or stream direction is arranged offset relative to the valve opening surface.

A more flexible packing collar can be provided. The flexible packing collar can be formed circumferentially around the valve device.

The anchoring device can have at least one of the following anchoring sections: a first anchoring section, which is covered with pericardium or a synthetic membranous plastic material, and a second anchoring section, which is free of pericardium and the synthetic membranous plastic material.

The anchor can be formed with a self-expanding anchor. The self-expanding anchor can have at least one anchor from the following group: self-expanding spiral element and self-expanding stent.

Some or all of the closing elements can consist of a biological material. Some of the closing elements can consist of a non-biological material. As an alternative, all closing elements can be made of a non-biological material.

### DESCRIPTION OF EMBODIMENTS

Below, additional embodiments are explained in greater detail with reference to the figures of a drawing. In this case:

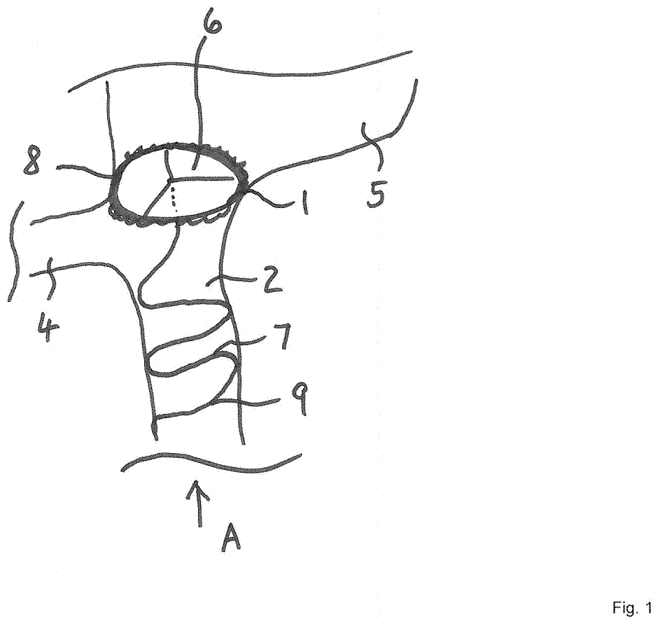

FIG. 1 shows a diagrammatic perspective depiction of a closing device that is implanted in the area of a vein;

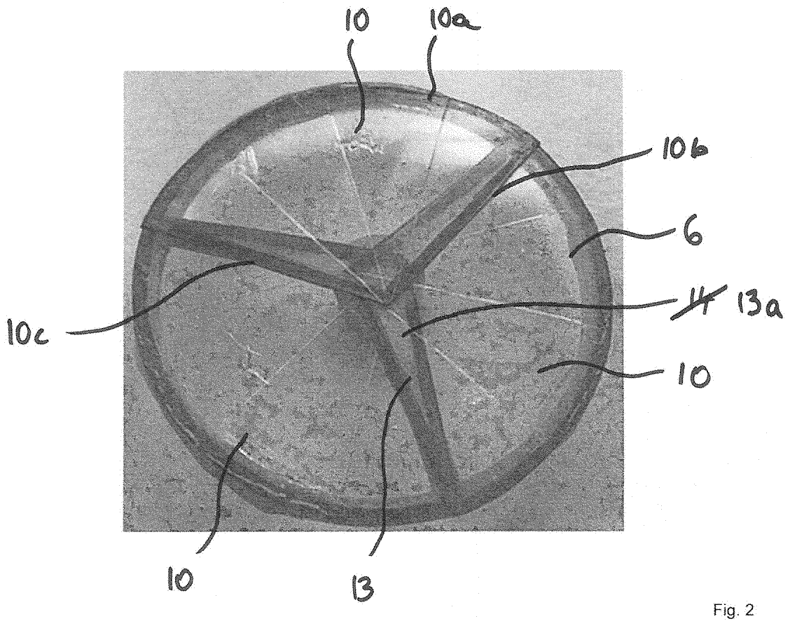

FIG. 2 shows a diagrammatic depiction of a valve device in a closed position from above, in which the closing elements rest on a support surface;

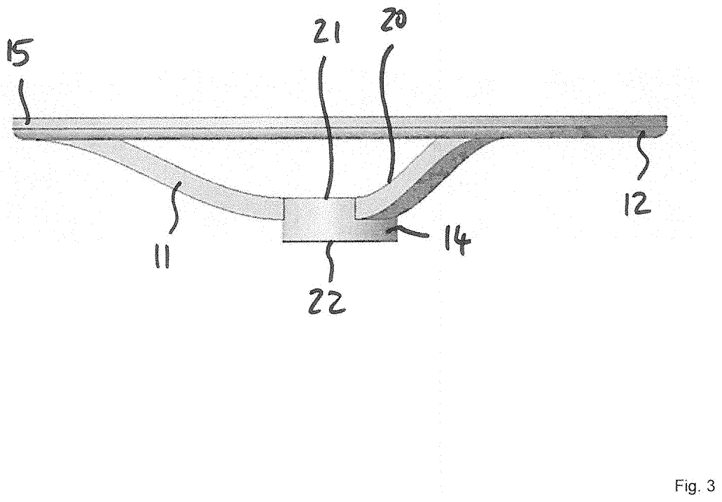

FIG. 3 shows a diagrammatic perspective depiction of a support structure for closing elements with a support designed in three dimensions;

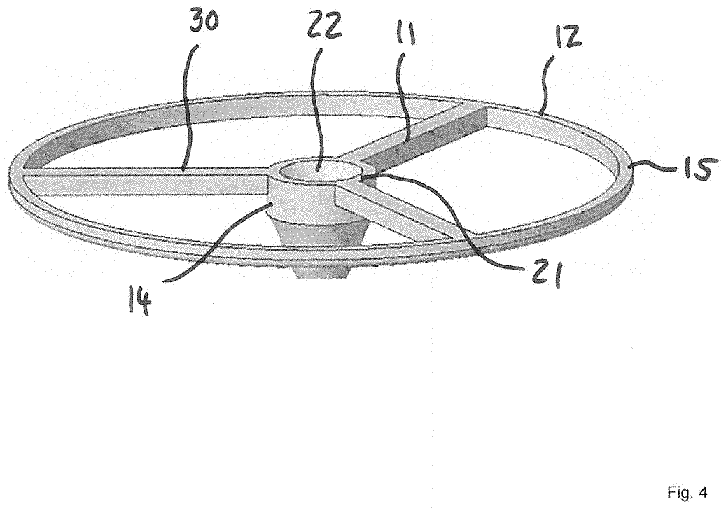

FIG. 4 shows a diagrammatic perspective depiction of a support structure with a support designed in two dimensions;

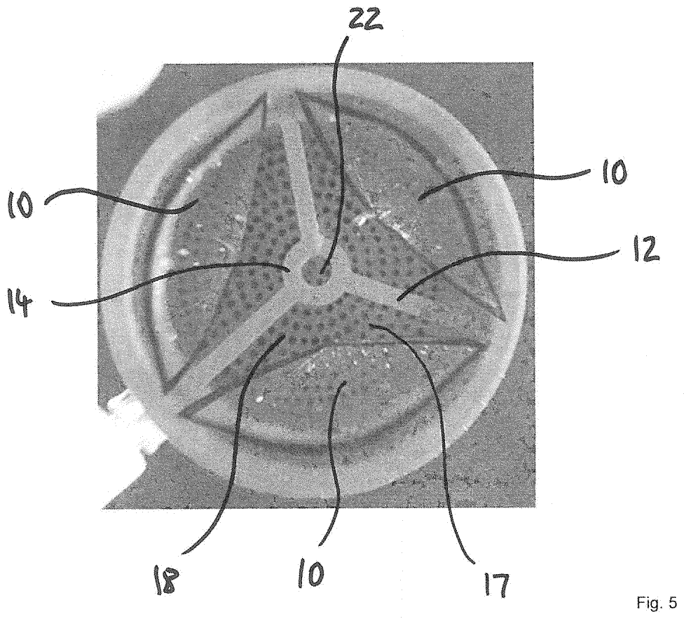

FIG. 5 shows a diagrammatic depiction of the valve device of FIG. 2 in an open position, in which the closing elements are arranged at least partially raised from the support structure;

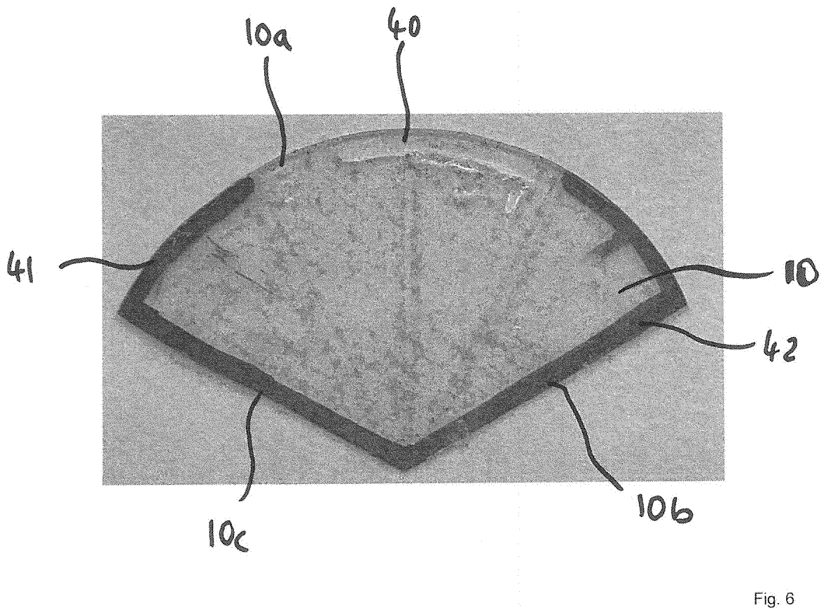

FIG. 6 shows a diagrammatic depiction of a closing element, which is attached along a part of the outer circular arc; and

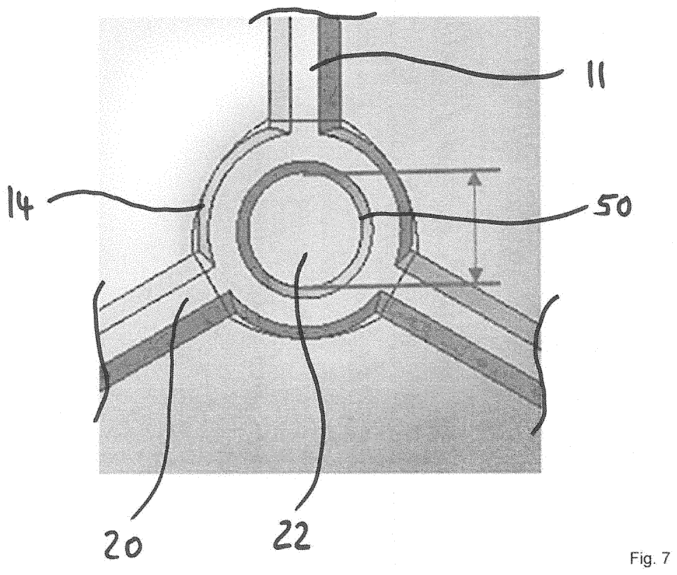

FIG. 7 shows a diagrammatic depiction of a section of the support structure with a flow nozzle and support elements starting therefrom in a star shape.

FIG. 1 shows a diagrammatic perspective depiction of a minimally-invasive implantable closing device 1 in the area of a vein 2. By means of the closing device 1, the flow into the vein can be opened and closed, wherein flow can take place only in the flow or stream direction that is shown in FIG. 1 by means of arrow A. In the opposite direction, the closing device 1 closes and thus prevents any backflow.

A section of a vena cava 3, a section of a hepatic vein 4, as well as a section of the right chamber of the heart 5 are shown.

The closing device 1 has a valve device 6, an anchoring device 7, as well as a flexible sealing ring 8, which is formed in a circumferential manner. In the embodiment that is shown, the anchoring device 7 is formed as a self-expanding anchor that has a spiral 9.

FIG. 2 shows a diagrammatic depiction of the valve device 6 in a closed position, in which no flow takes place, from above. In the closed position that is shown, closing elements 10 rest on support elements 11 of a support structure 12, for which FIGS. 3 and 4 show different embodiments. In this case, the closing elements 10 come to rest in overlapping areas 13 on the support elements 11, in which edge-side sections 13a of the closing elements 10 overlap. In the embodiment shown, the closing elements 10 are formed as sectors that are bounded in each case by a circular arc 10a and two radii 10b, 10c. In the area of the radii 10b, 10c, the overlapping areas 13 are made between adjacent closing elements 10. In the embodiment shown, the sectors of the closing elements 10 in each case comprise an area of somewhat more than 120 degrees in order to form the overlapping areas 13.

The support elements 11 of the support structure 12 extend in a star shape starting from a central area 14 and are arranged in a circumferential manner at equal intervals. The support structure has a circumferential edge 15 (cf. in particular FIGS. 3 and 4).

FIGS. 3 and 4 show diagrammatic perspective depictions of various embodiments for the support structure 12. For the same features, the same reference numbers are used in FIGS. 3 and 4. In a first embodiment of the support structure 12 according to FIG. 3, a support 20 designed in three dimensions is formed for the closing elements 10. An opening surface 21 of an indentation 22 in the central area 14 in the stream or flow direction is arranged offset relative to the valve opening surface, which is surrounded by the circumferential edge 15. FIG. 4, however, shows an alternative embodiment of the support structure 12, in which a flat support 30 designed in two dimensions is provided for the closing elements 10.

FIG. 5 shows a diagrammatic perspective depiction of the valve device 6 in an open position, in which the closing elements 10 are raised (swung open) at least in the central area 14 by the support elements 11 of the support structure 12, so that the indentation 22 as well as additional indentation areas 17 of the support structure 12 are released, and flow can take place.

According to the exemplary embodiment in FIG. 5, a sieve insert 18 is provided in the area of the valve opening, which can be connected detachably or non-detachably to the support structure 12.

In the embodiment that is shown, the closing elements 10 are designed as non-surface-rigid closing elements that bend upward in order to release the flow. To this end, the closing elements 10 consist of, for example, a plastic material.

The closing elements 10 can be arranged on the support structure 12 by means of a joint connection, for example by means of gluing or welding. FIG. 6 shows diagrammatically a closing element 10, which is designed as a sector. The closing element 10 is fastened to the support structure 12 along a connecting area 40, which extends along the circular arc 10a. Outside of the connecting area 40, the closing element 10 is free of a connection with the support structure 12, in particular even in corner areas 41, 42, in which the radii 10b, 10c as well as the circular arc 10a of the sector meet.

FIG. 7 shows a cutaway of the support structure 12 in the central area 14 with the indentation 22, which in the embodiment that is shown is formed with a flow nozzle 50. Starting from the flow nozzle 50, support elements 11 extend in a star shape. In the depicted configuration, the support elements 11 are tapered, which elements are designed as, e.g., a carrier that operates the support 20.

The features disclosed in the description above, the claims, as well as the drawing can be important both individually and in any combination for the implementation of the various embodiments.

1. Arrangement of a minimally-invasive implantable closing device (1) in the superior or inferior vena cava of a human body, with:
a valve device (6), having
closing elements (10), which extend flat over a respective joint surface and in each case can be moved between a closed position, in which the closing elements (10) together close a valve opening, and an opening position, in which a flow is released through the valve opening; and
a support structure (12), which is arranged in the area of the valve opening and is formed with support elements (11) and at least one indentation (20), wherein the closing elements (10) in the closed position
rest at least partially on the support elements (11), and in the open position, they are separated at least partially from the support elements (11) and thus release the indentation (20) to allow flow; and
an anchoring device (7), which has an anchor and is set up to anchor the valve device (6) in the area of the superior or inferior vena cava adjacent to the vein opening in the right chamber of the heart.

2. Arrangement according to claim 1, characterized in that adjacent closing elements (10) overlap in edge-side sections (13a) at least in the closed position, and the support elements (11) extend at least along the overlapping edge-side sections (13a).

3. Arrangement according to claim 1, characterized in that the valve device (6) is formed as a one-way valve.

4. Arrangement according to claim 1, characterized in that the closing elements (10) in each case comprise a sector surface, wherein adjacent closing elements overlap along the radii (10b, 10c) laterally bounding the sector surface.

5. Arrangement according to claim 4, characterized in that the closing elements (10) in each case comprise a sector of at least approximately 90 degrees.

6. Arrangement according to claim 1, characterized in that the closing elements (10) are fastened at least in sections along an outside circumferential edge in such a way that a section of the closing element (10) that is radially inside relative to the circumferential edge can swing open into the open position and can thus release the flow.

7. Arrangement according to claim 1, characterized in that the indentation (22) of the support structure (12) is formed with a flow nozzle (50).

8. Arrangement according to claim 1, characterized in that the support structure (12) has support elements (11), which extend in a star shape.

9. Arrangement according to claim 1, characterized in that the support structure (12) supports the closing elements (10) in the closed position in an essentially two-dimensional flat plane.

10. Arrangement according to claim 1, characterized in that the support structure (12) supports the closing elements (10) in the closed position in a curved three-dimensional plane.

11. Arrangement according to claim 1, characterized by a flexible packing collar (8).

12. Arrangement according to claim 1, characterized in that the anchoring device (7) has at least one of the following anchoring sections:
a first anchoring section, which is covered with pericardium or a synthetic membranous plastic material, and
a second anchoring section, which is free of pericardium and the synthetic membranous plastic material.

13. Arrangement according to claim 1, characterized in that the anchor is formed with a self-expanding anchor.

14. Arrangement according to claim 1, characterized in that some or all of the closing elements (10) consist of a biological material.

15. Minimally-invasive implantable tricuspid valve prosthesis, with an arrangement according to claim 1.
